(19) United States
(12) Patent Application Publication
(10) Pub. No.: US 2014/0048730 A1
(43) Pub. Date: Feb. 20, 2014

(54) Radiation Protection System

(71) Applicant: ECO CATH-LAB SYSTEMS, INC., Emeryville, CA (US)

(72) Inventors: Jim Niedzielski, Linden, MI (US); Richard Weaver, Linden, MI (US)

(73) Assignee: ECO CATH-LAB SYSTEMS, INC., Emeryville, CA (US)

(21) Appl. No.: 13/837,773

(22) Filed: Mar. 15, 2013

Related U.S. Application Data
(60) Provisional application No. 61/683,320, filed on Aug. 15, 2012.

(51) Int. Cl. A61B 6/10

(52) U.S. Cl. CPC A61B 6/107
USPC 250/519.1

(57) ABSTRACT

A radiation protection system for protecting medical personnel from radiation being applied from a radiation source to a patient positioned on a table that includes a radiation-shielding wall including, upper shield suspended by a gas spring lift arm, the upper shield consisting of translucent radiation resistance window, with a left and right side of flexible radiation shielding material, that telescopes down on each side of the table to form a complete radiation barrier. The shield is positioned above the table. The shield also has a radiation-shielding flexible interface attached to the radiation shielding window that covers a portion of the patient.

### FIELD OF THE INVENTION

The present invention claims priority to U.S. Provisional Application No. 61/683,320 filed on Aug. 15, 2012, the entirety of which is also incorporated herein by reference to the extent permitted by law. This invention relates generally to radiation shielding devices and, more particularly, to a radiation protection system for use in a medical procedure to protect medical personnel from the harmful effects of radiation. Further, in an embodiment of the invention, the radiation protection system allows medical procedures to be performed using x-ray radiation to create internal images of a patient without the use of personal protective lead aprons.

### BACKGROUND OF THE INVENTION

X-rays are used in a wide variety of medical procedures, many of which require medical personnel to be in direct contact with the patient, thereby exposing such personnel to radiation. When working with a patient on an x-ray table, doctors and other medical personnel can be exposed to primary radiation that emanates directly from the source or can be exposed to secondary radiation that is scattered by an object such as the x-ray detector, the x-ray table, and even the patient. For this reason, both fixed and mobile lead shields are employed in fluoroscopic procedures to minimize radiation exposure. Such shields are constructed of some form of radiation resistant material in many configurations that are interposed between the operators and the radiation source.

Despite the use of these shields, medical personnel are still exposed to radiation. It is therefore imperative that such personnel wear leaded protective clothing (including full lead aprons, vests, thyroid collars and leaded glasses). In addition, the doctors performing these radiologic procedures typically spend many hours per day, several days per week over many years throughout their medical careers in such procedures. This long term, cumulative exposure may cause adverse effects. Furthermore, the wearing of heavy lead aprons may have long term deleterious effects resulting in disabling disorders of the spine in a significant number of operators.

There are some existing shields for protecting and shielding against radiation in x-ray laboratories in the prior art. These prior arts disclose shields made of radiation resistant material that are either mobile or attached to the x-ray table, ceiling or a separate structure in the room and can be adjusted between the operators and the x-ray source. Though there are numerous shapes and designs for these shields, and although they may be constructed of various materials, they do not sufficiently protect against radiation exposure, and medical personnel must still wear heavy and encumbering leaded protective clothing.

It is in view of the above that the present inventions were developed. Among the objects and features of the inventions are reducing the radiation exposure of staff and medical personnel in an x-ray laboratory or during a medical procedures.

An object of one embodiment of the invention is substantially reducing primary radiation around an x-ray table and thereby permitting doctors to perform fluoroscopic based medical and surgical procedures with access to a patient without being exposed to excessive amounts of radiation.

An object of one embodiment of the invention is reducing secondary radiation in the region around an x-ray table where doctors operate on a patient.

In one embodiment of the present invention, a radiation protection system around an x-ray table is provided with a radiation-shielding wall, a radiation-shielding window, and a radiation-shielding flexible interface is attached to the wall and over the patient on the x-ray table. The wall separates an x-ray emitter from an operating region where doctors and other medical personnel are in close proximity to a patient on the x-ray table. The radiation shielding system is attached to the x-ray table in the operating region such that the shield is interposed between the radiation source and the medical personnel. The system is suspended from the ceiling by a lift or arm readily known to persons of ordinary skill in the art and interfaces with the x-ray table with at least one connection point, so that the wall can move in conjunction with the table. To complete the radiation shielding system, affixed to the translucent leaded window in the center of the system is a flexible radiation resistant drape that covers at least a portion of the patient and completes the barrier between the operators and the radiation source. At least one access port may be formed in the radiation resistant drape, and at least one radiation-shielding cloak can cover the access port and surround medical instruments that are threaded through the port and inserted into the patient.

Other systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

### SUMMARY OF THE INVENTION

Additional features and advantages of an embodiment will be set forth in the description which follows, and in part will be apparent from the description. The objectives and other advantages of the invention will be realized and attained by the structure particularly pointed out in the exemplary embodiments in the written description and claims hereof as well as the appended drawings.

In one embodiment of the invention the radiation protection system includes a radiation-shielding wall including a radiation-shielding window in-between a left upper shield and a right upper shield, and a radiation-shielding flexible interface attached to the radiation-shielding window.

In another embodiment of the invention, a portion of each of the left and right upper shields is connected to a corresponding portion of a lower wall.

In another embodiment of the invention a radiation-shielding interface is connected to the radiation-shielding window.

In another embodiment of the invention, the radiation-shielding window has a patient hoop located at or towards the bottom portion of the radiation-shielding window.

In another embodiment of the invention, the patient hoop extends along entire the radiation-shield window.

In another embodiment of the invention the radiation protection system includes a radiation-shielding wall including a radiation-shielding window in-between a left upper shield and a right upper shield, and a radiation-shielding flexible interface is attached to the radiation-shielding window. In this embodiment the radiation-shielding window is movably attached to the left and right upper shields.

In another embodiment of the invention, the radiation protection system includes an upper structure, a radiation-shielding wall including an adjustable radiation-shielding window in-between a left upper shield and a right upper shield, and a radiation-shielding flexible interface is attached to the radiation-shielding window. In this embodiment, the radiation-shielding window is suspended from the upper structure by first and second horizontal rods; each of the first and second horizontal rods attached a corresponding vertical rod located at each side of the radiation-shielding window.

In another embodiment of the invention, the vertical rods are configured to move as the radiation-shielding window is adjusted.

In another embodiment of the invention, a lower shield is attached to the left and right upper shields.

In another embodiment of the invention the lower shield is configured to contract upwards into the upper shield and lower downwards.

Other systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

### BRIEF DESCRIPTION OF THE DRAWINGS

The accompanying drawings, which are incorporated in and constitute a part of this specification, illustrate an implementation of the present inventions and, together with the description, serve to explain the advantages and principles of the inventions. In the drawings:

FIG. 1 illustrates a perspective view of a radiation protection system according to one embodiment of the invention.

FIG. 2 illustrates a right side table shield that is deployed.

FIG. 3 illustrates a right side lower shield that is deployed.

FIG. 4 illustrates an under table shield.

FIG. 5 illustrates a table to shield connection mechanism.

FIG. 6 illustrates a shield to table adjustment mechanism.

FIG. 7 illustrates a rail clamp for an auxiliary rail mount.

FIG. 8 illustrates a table to shield adjustment mechanism in a retracted position.

FIG. 9 illustrates an adjustable window in a tilted position.

FIG. 10 illustrates an adjustable window in a neutral position.

FIG. 11 illustrates an adjustable window in a tilted and raised position.

FIG. 12 illustrates the upper portion of a patient drape.

FIG. 13 illustrates the lower portion of a patient drape.

FIG. 14 illustrates a mounting mechanism for the radiation protection system.

FIG. 15 illustrates the radiation protection system detached for deployment/storage.

FIG. 16 illustrates the connection of the translucent window to the structure

### DETAILED DESCRIPTION OF THE INVENTION

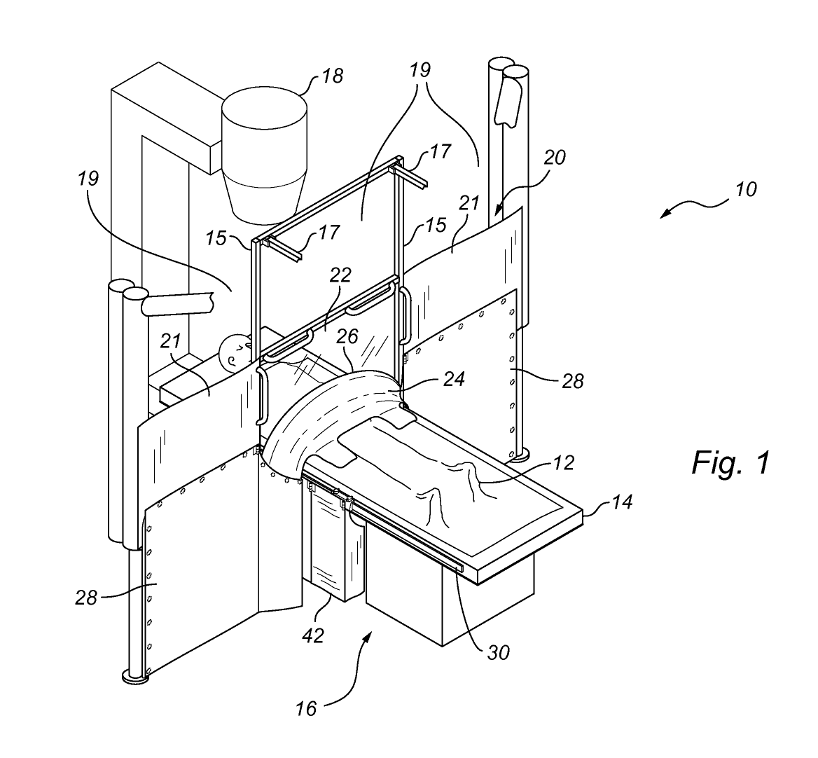

Referring to the accompanying drawings in which like reference numbers indicate like elements, FIG. 1 illustrates a radiation protection system 10 that separates the medical personnel operating region 16 from the x-ray emitter 18. The patient 12 is on the x-ray table 14. The radiation protection system 10 includes a radiation-shielding wall consisting of an upper shield 20, which serves as a barrier between the radiation emitter and the medical personal. The radiation protection system 10 may be suspended from a ceiling or overhead surface. In some cases the radiation protection system may be suspended by a gas spring lift arm 64 attached to the ceiling or rails, or by other suitable means known to those of ordinary skill in the art. Some or all of the components of the radiation protection system may be maneuvered manually, may have a mechanical control which permits the shield to be operated via a control mechanism, or may be able to operate both manually or via a control mechanism. The mechanical control and control mechanisms are of the type well known to those or ordinary skill in the art. For example, the gas spring lift arm 64 and the upper shield may be adjusted or moved manually or may have a control mechanism. The upper shield may include a radiation shielding window 22 in between a left upper shield and a right upper shield 21. The left and right upper shields may be made of a blocking material on both sides, such as leaded vinyl or the like. The radiation shielding window 22 may also be made of a blocking material such as a leaded acrylic. The lower shield 28 is incorporated into the corresponding left or right upper shield 21. Once the shield 20 is positioned over the patient, then the lower shield 28 can be deployed or lowered and connected to the table by any means known to those skilled in the art. A radiation-shielding interface or patient drape 24 is connected to the radiation-shielding window 22. A portion of the radiation-shielding interface extends over at least a portion of the patient 12 on the x-ray table 14.

The radiation-shielding wall or upper shield 20 is constructed from well-known radiation-blocking materials such as lead, leaded vinyl, leaded acrylic or the like and the window 22 can be made of a transparent material, thereby permitting visual contact between medical personnel (not shown) in the operating region 16 and the patient head 12.

In one embodiment of the invention, the radiation-shielding window has a patient hoop 26 attached to the bottom portion of the radiation-shielding window 22. As the radiation shield-shielding window 22 is tilted towards the patient's feet, the patient hoop 26 raises to accommodate access to the patient as well as maintain a radiation protection seal. The patient hoop 26 may extend the entire horizontal length or a portion of the radiation shield window to accommodate access to the patient.

The radiation-shielding window 22 is movably attached to the upper shield 20. The radiation-shielding window 22 may be connected to the left and right upper shields 21 on both sides by a flexible radiation resistant material and suspended from the upper structure of the radiation protection system 10 by two horizontal rods 17 which attach to two rods 15 one on each side of the radiation-shielding window. The rods 15 extend in a downward direction from the horizontal rod 17. The rods 15 are configured to move as the radiation-shielding window top 22 is adjusted towards the patient's feet, for example the radiation-shielding window top can be tilted at various degrees, for example from 0 (straight up or neutral) to 30 caudal. More specifically, the radiation-shielding window 22 is connected on each side of the lower portion 22 of the radiation-shielding window 22 to the left and right upper shields by rods that manually move the window up the rods 15 as the window is tilted towards the patient's feet in order to attain the extreme angles needed from the x-ray emitter during a procedure as well as make it possible to achieve access to the groin and wrist area of the patient while maintaining a complete radiation free environment for the medical personnel. A portion of the lower shield 28 consists of left and right shields that can telescope down or retract upwards into the upper shield 20 when the system is to be stored or deployed. The shield may be maneuvered manually or may have a mechanical control which permits the shield to be operated via a control mechanism. The mechanical control and control mechanisms are of the type well known to those or ordinary skill in the art. Once the radiation protection system 10 is in place over the patient, the shields 28 can be lowered to complete part of the lower radiation barrier as illustrated in this FIG. 1.

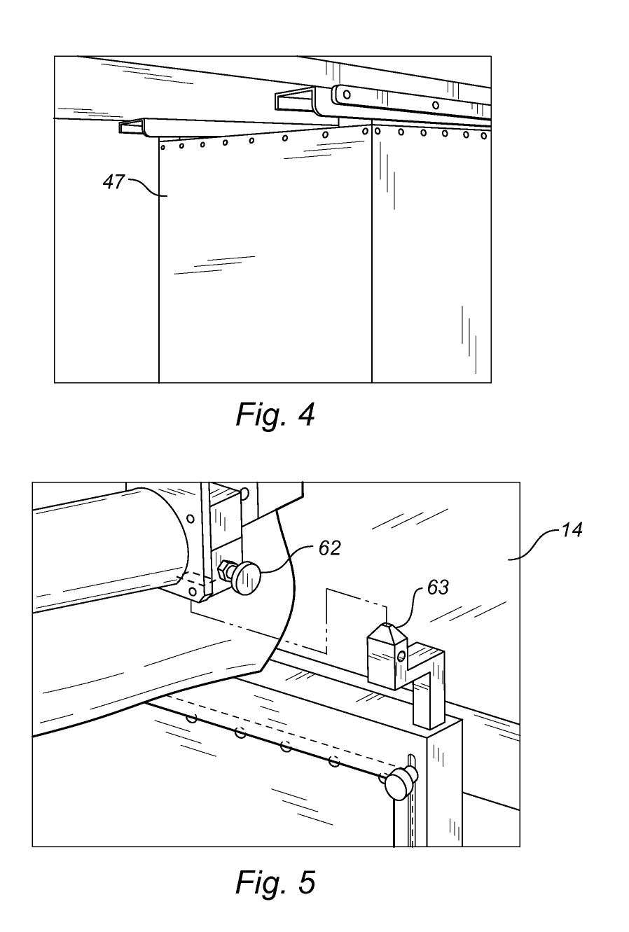

Additionally, a curtain shield 42 is attached along the x-ray table accessory rail 30 along with the lower shield side drapes 28 when they are in the deployed position and an x-ray table shield under the x-ray table 47, which is shown in FIG. 4. To complete the radiation barrier. If needed, magnets or other suitable connection means can be attached to the shields/drapes in order to make a definite connection. It will be evident to those skilled in the art that the lower shield and curtain shields 42 could be constructed from various panels or segments and how these panels and segments can be connected.

As shown in FIG. 5 connecting/disconnecting the upper shield to the x-ray table 14 is accomplished by attaching/detaching the upper shield 20 from the shield to table connection points 62 and 63, 62 being the female end fixed to the upper shield with a quick attachment/release pin incorporated and 63 the male end fixed to the table, extending/retracting the lower shield side drapes 28 and lowering/raising the upper shield 20, then moving the upper shield 20 up and away from the patient.

With the radiation protection system 10 set in place, doctors and other medical personnel in the operating region 16 are shielded from the x-ray emitter 18 and x-ray scatter during radiologic procedures. The radiation protection system 10 separates the operating region 16 from the x-ray emitter 18 to protect the doctors and medical personnel from exposure to most primary as well as scatter radiation from the x-ray emitter 18.

As shown in FIG. 11, the radiation-shielding patient drape 24 is interposed between the medical personnel and the patient 12 to protect against most x-ray scattering from the patient 12 and the x-ray table 14. The radiation-shielding flexible patient drape 24 covers the opening of the patient hoop 26 in the upper shield 20 and joins the radiation wall with the x-ray table 14 and the radiation shielding window 22 to protect-against most radiation leaking into the operating region 16 when the x-ray table is moved.

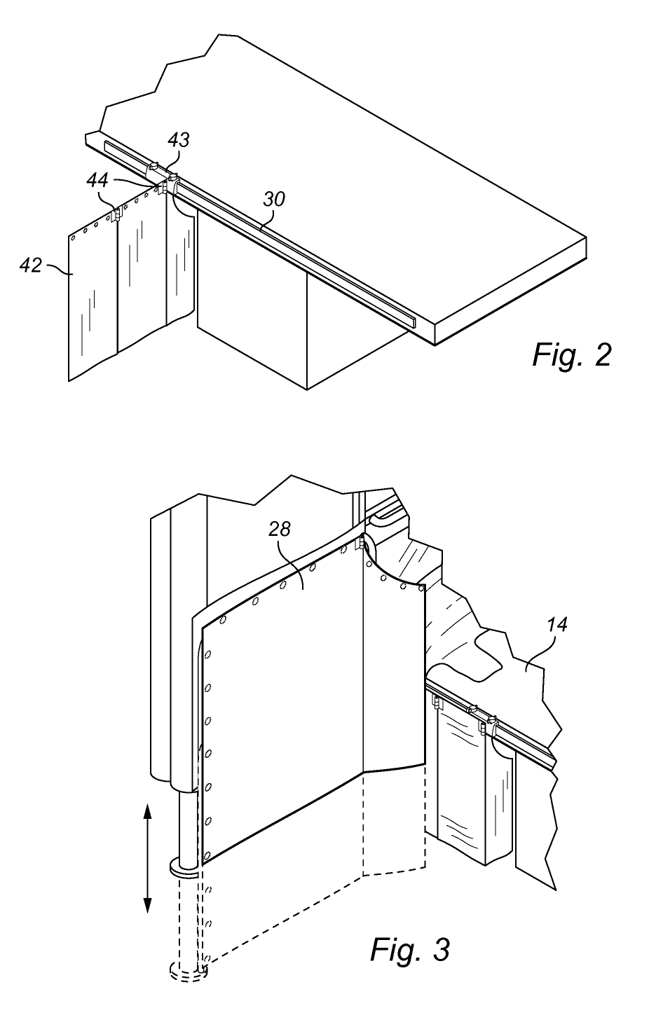

FIGS. 2 and 3 illustrate the table side curtain shields 42 attached along an accessory rail 30. The connection means 43 allows the curtain shield position to be adjusted along the table length. The hinge 44 located on the table side curtain shield 42 permits the table side curtain shield 42 position to be adjusted along the side of the table and to the extendable lower shield portion 28 by extending or collapsing the curtain shield against lower shield portion 28 as necessary.

FIG. 4 illustrates a portion of the lower shield 47 positioned under the table, to form a complete radiation barrier with the upper shield and table side curtain shield. The lower shield 47 extends downwards from the table to the floor.

FIG. 5 illustrates the embodiment of the table to radiation protection system 10 via the shield to table connection points 62 and 63. This connection allows the radiation protection system 10 to stay connected to the table. Thus allowing the radiation protection system 10 to follow the motion and movements as the table 14 is moved.

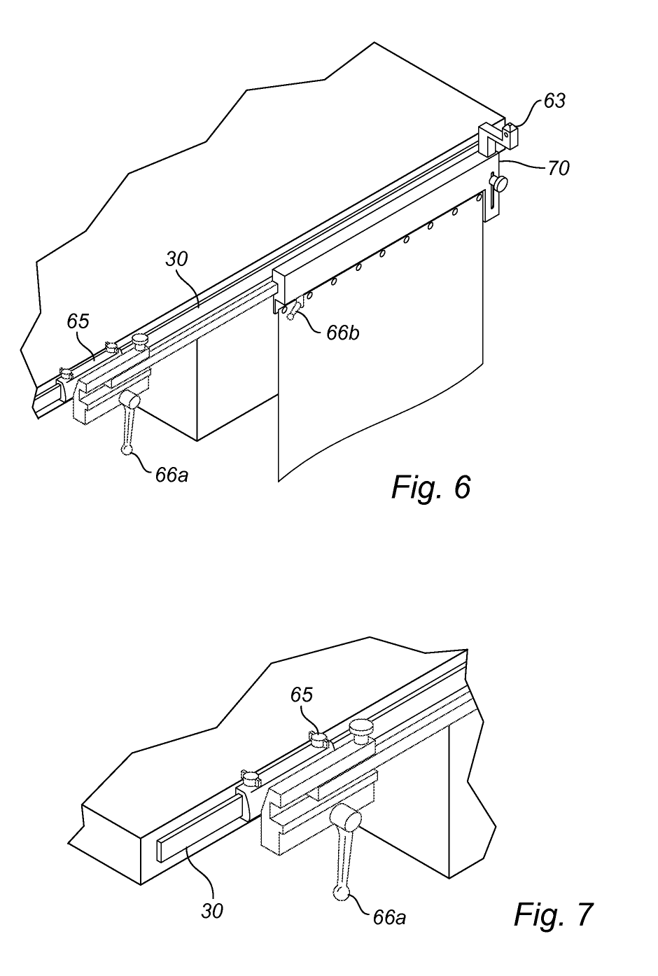

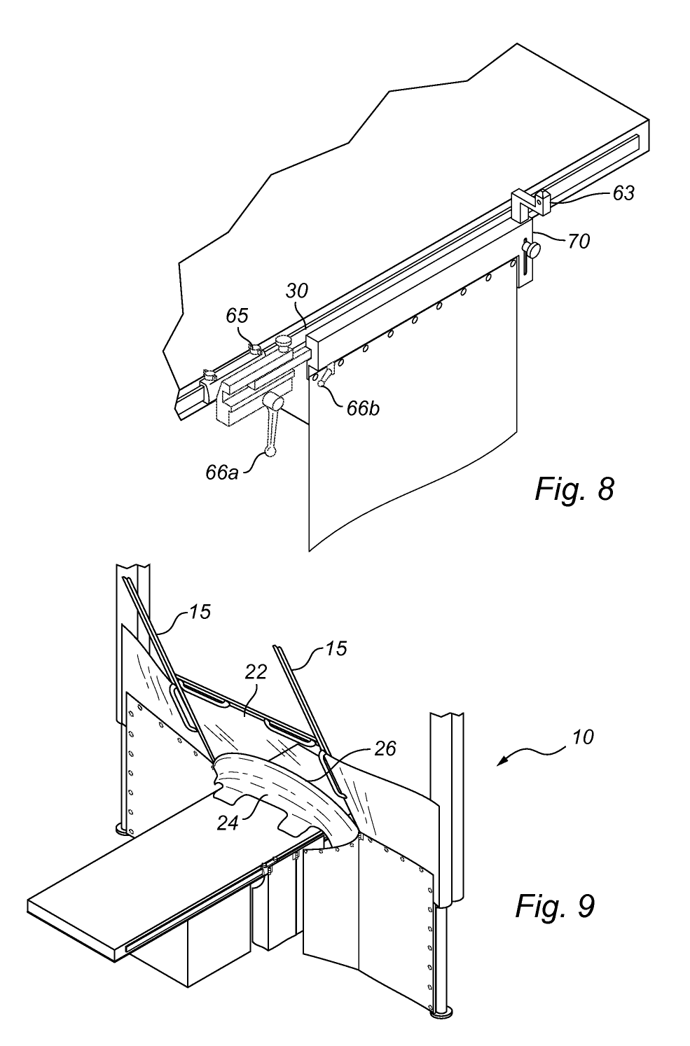

FIGS. 6, 7 and 8 illustrate the mechanisms 70, 65, 66a, 66b, and 63 that permit the various adjustments and connections required for positioning the radiation protection system 10 to the table as well as the various patient anatomies and angles of the x-ray emitter 18. A table side rail 65 is attached to the table accessory rail 30. Additionally, a table/shield connection adjustment mechanism 70 is attached to the table side rail 65, which allows for the table adjustment mechanism 70 to adjust radiation protection system's position horizontally along the table. Once the appropriate positioning is achieved, a lock or clamp 66a can be used to maintain the position of the adjustment mechanism 70 and a lock or clamp 66b can be used to maintain the position of the table side rail 65. If the position of the radiation protection system needs to be altered, the lock can be unlocked or the clamp can be released and the radiation protection system can be adjusted.

The combination of these adjustment mechanisms allow for adjusting the radiation protection system 10 to various patient sizes of anatomy. Thus maintaining at all times a complete radiation barrier.

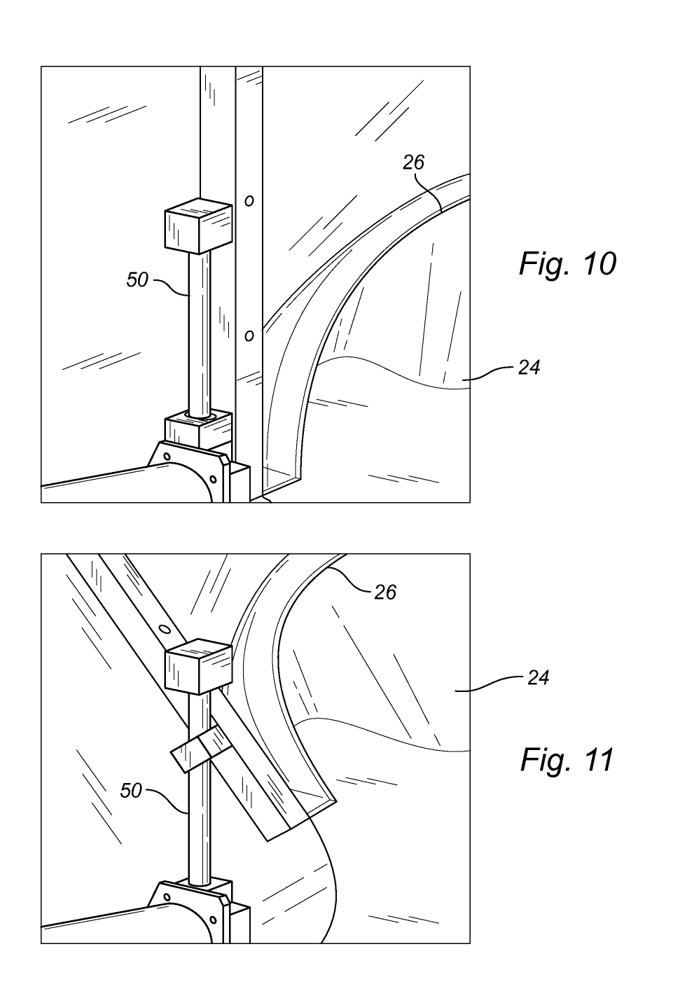

FIGS. 9, 10, and 11 illustrate the translucent radiation resistant window 22 in conjunction with the patient hoop 26, patient drape 24 and the novel approach for achieving access to the patient's groin and or wrist during an interventional procedure for example for the purpose of inserting a catheter while performing fluoroscopy. The lower part of the radiation resistant window 22 is attached to the radiation protection system 10 frame by a rod 50 on each side of the window. When the radiation protection window is in the neutral position (straight up and down) the patient hoop is at its smallest opening. As the top of the radiation protection window 22 is moved towards the patient's feet so as to achieve images of the lower anatomy the bottom of the radiation protection window 22 rises straight up along the rods 50 for access to the patient's groin and/or wrist area while maintaining a radiation barrier at all times.

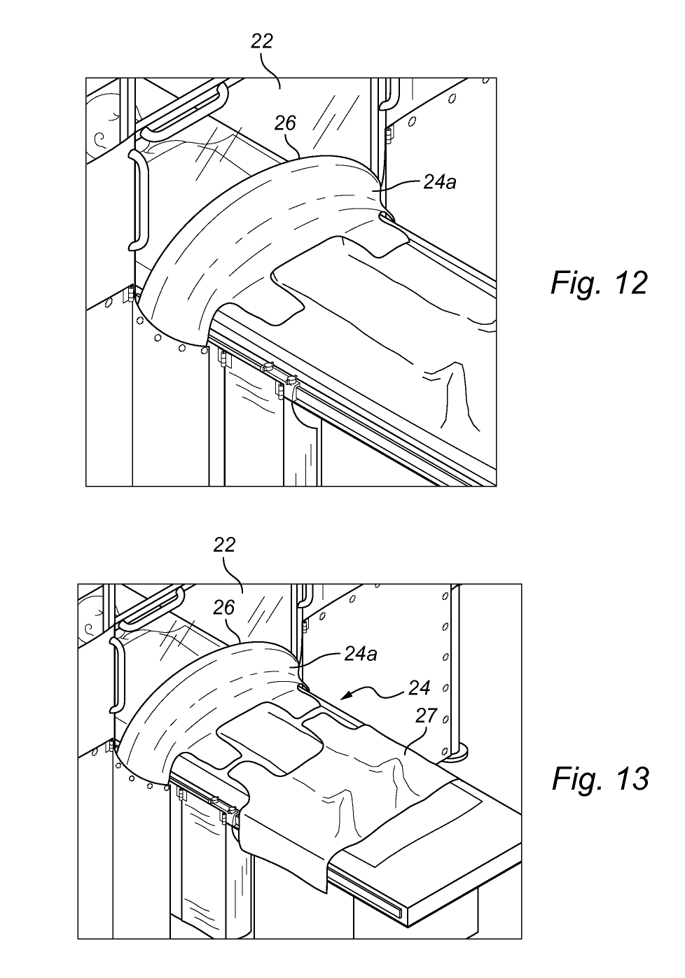

FIGS. 12 and 13 illustrate an embodiment of the flexible patient upper drape 24 that can be attached to or secured to the radiation shielding window 22. This embodiment of the flexible patient upper drape affords access to the left or right femoral area or radial area of the patient This particular embodiment of the flexible patient drape 24 consists of two parts, a first part 24a is attached to the radiation-shielding window contains two site openings. The second part, the patient lower drape 27 is laid over the patient and has covering flaps, each of which may or may not be magnetically attached over their respective site or folded out of the way for access.

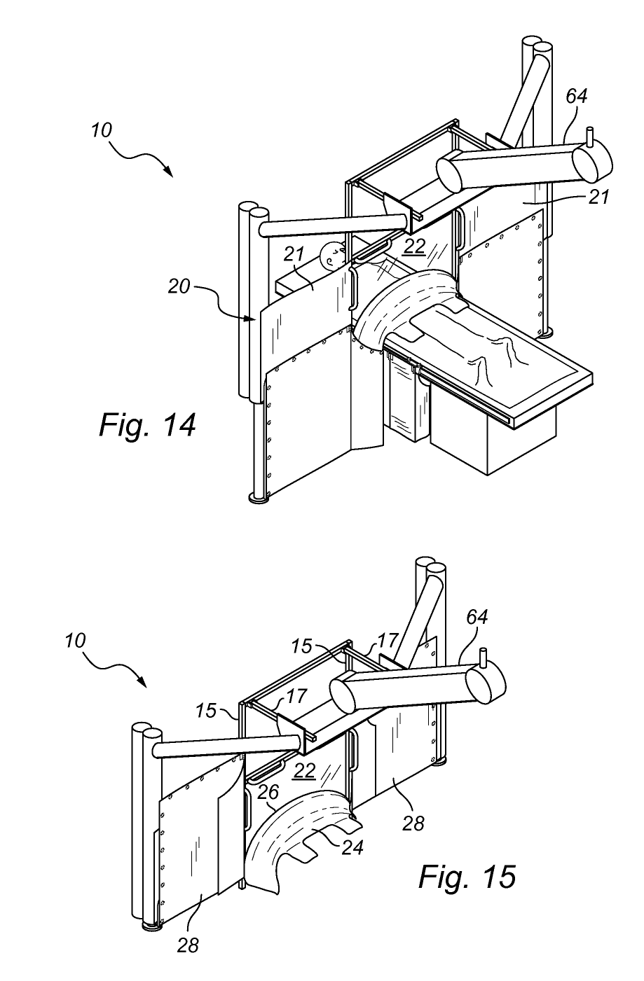

FIG. 14 illustrates the shield 10 being suspended by a gas spring lift arm 64 which is attached to the ceiling super structure. The lift 64 improves the degrees of motion of the upper shield and has a smaller footprint in the ceiling. This in turn helps reduce the overall size and weight of the radiation protection system.

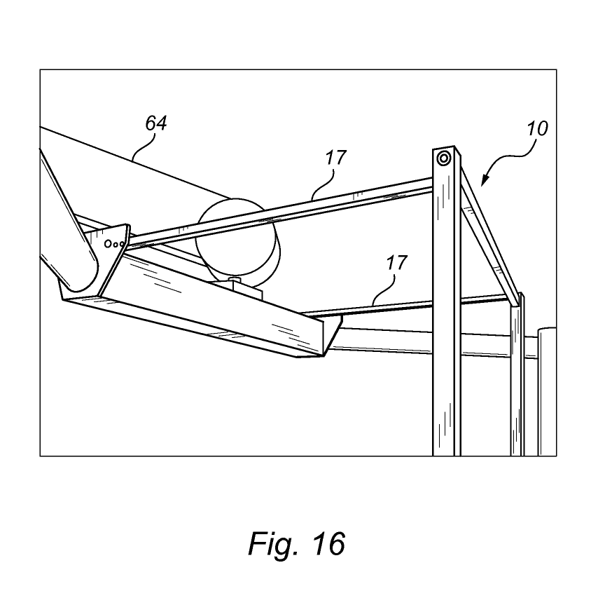

FIGS. 15 and 16 illustrates the radiation protection system 10 when it is in its stored position. In this figure, the lower shield side shields 28 are retracted into the upper shield, the patient drapes 24, the patient hoop 26, the two upper rods 15 connecting the translucent radiation protection window to two horizontal rods 17 that are attached to the upper shield and the translucent radiation protection window 22. In this configuration, the radiation protection system can be stored for later use or prepared for a procedure.

In the case of a procedure, the medical personnel would use a proprietary sterile drape (not shown) to drape the upper shield 20. The patient can be prepped for the medical procedure while lying on the table. The upper shield 20 can then be maneuvered into place over the abdomen of the patient. Once the upper shield 20 is connected to the table 62 and 63 (the shield to table connection coupler), the lower shield side shields 28 will be extended down towards the floor and the table side shields 42 will be moved into place along the side of the table.

While various embodiments of the present invention have been described, it will be apparent to those of skill in the art that many more embodiments and implementations are possible that are within the scope of this invention. Accordingly, the present invention is not to be restricted except in light of the attached claims and their equivalents.

1. A radiation protection system comprising:
a radiation-shielding wall including a radiation-shielding window in-between a left upper shield and a right upper shield, and
a radiation-shielding flexible interface attached to the radiation-shielding window.

2. The radiation protection system according to claim 1, wherein a portion of each of the left and right upper shields is connected to a corresponding portion of a lower wall.

3. The radiation protection system according to claim 1 wherein a radiation-shielding interface is connected to the radiation-shielding window.

4. The radiation protection system according to claim 1 wherein the radiation-shielding window has a patient hoop located at or towards the bottom portion of the radiation-shielding window.

5. The radiation protection system according to claim 4 wherein the patient hoop extends along entire the radiation-shield window.

6. A radiation protection system comprising:
a radiation-shielding wall including a radiation-shielding window in-between a left upper shield and a right upper shield, and
a radiation-shielding flexible interface is attached to the radiation-shielding window,
wherein,
the radiation-shielding window is movably attached to the left and right upper shields.

7. A radiation protection system comprising:
an upper structure;
a radiation-shielding wall including an adjustable radiation-shielding window in-between a left upper shield and a right upper shield, and
a radiation-shielding flexible interface is attached to the radiation-shielding window,
wherein,
the radiation-shielding window is suspended from the upper structure by first and second horizontal rods, each of the first and second horizontal rods attached a corresponding vertical rod located at each side of the radiation-shielding window.

8. The radiation protection system according to claim 7 wherein the vertical rods are configured to move as the radiation-shielding window is adjusted.

9. The radiation protection system according to claim 7 wherein a lower shield is attached to the left and right upper shields.

10. The radiation protection system according to claim 9 wherein the lower shield is configured to contract upwards into the upper shield and lower downwards.
